(19) United States
(12) Patent Application Publication
(10) Pub. No.: US 2024/0029898 A1
(43) Pub. Date: Jan. 25, 2024

(54) MEDICAL INFORMATION PROCESSING DEVICE, MEDICAL INFORMATION PROCESSING METHOD, AND STORAGE MEDIUM

(71) Applicant: CANON MEDICAL SYSTEMS CORPORATION, Otawara-shi (JP)

(72) Inventors: Yasuko FUJISAWA, Nasushiobara (JP); Takuya SAKAGUCHI, Utsunomiya (JP)

(73) Assignee: CANON MEDICAL SYSTEMS CORPORATION, Otawara-shi (JP)

(21) Appl. No.: 18/353,135

(22) Filed: Jul. 17, 2023

(30) Foreign Application Priority Data
Jul. 21, 2022 (JP) 2022-116304
Jul. 21, 2022 (JP) 2022-116628

(51) Int. Cl. G16H 50/70; G16H 40/20; G06Q 40/08

(52) U.S. Cl. CPC G16H 50/70; G16H 40/20; G06Q 40/08

(57) ABSTRACT

A medical information processing device of an embodiment includes processing circuitry. The processing circuitry acquires a first attribute factor group which is a plurality of attribute factors relating to a disease of a target patient and which is a plurality of attribute factors at a first timing. The processing circuitry estimates a second attribute factor group which is a plurality of attribute factors at a second timing after the first timing on the basis of the first attribute factor group. The processing circuitry outputs information including the first attribute factor group and the second attribute factor group via an output interface.

### CROSS-REFERENCE TO RELATED APPLICATION

The present application claims priority based on Japanese Patent Application No. 2022-116304 and 2022-116628, filed Jul. 21, 2022, the contents of which is incorporated herein by reference.

### FIELD

Embodiments disclosed in the present specification and drawings relate to a medical information processing device, a medical information processing method, and a storage medium.

### BACKGROUND

The selection of a treatment method is decided to some extent according to a state of a patient's life faculties and a disease degree by guidelines and the like. However, it is difficult to predict the prognostic states of patients to which treatment has been applied. Although the prognosis requires the evaluation of the basic physical strength of a patient, states of organs other than the disease, and an immune function as well as the determination of the state of a diseased organ or a tumor, relationships therebetween are not clear. Also, it is preferred to ascertain the state of an organ or tumor in which the disease has occurred and it is not sufficient to ascertain the basic physical strength of the patient, states of organs other than the disease, the immune function, and the like. Thus, patients and their family members may not be able to select treatment strategies that improve outcomes.

### BRIEF DESCRIPTION OF THE DRAWINGS

FIG. 1 is a diagram showing an example of a constitution of a medical information processing system in a first embodiment.

FIG. 2 is a diagram showing an example of a configuration of a terminal device in the first embodiment.

FIG. 3 is a diagram showing an example of a configuration of a medical information processing device in the first embodiment.

FIG. 4 is a flowchart showing a flow of a series of processing steps of processing circuitry according to the first embodiment.

FIG. 5 is a diagram for describing a stratification process.

FIG. 6 is a diagram showing an example of an attribute distribution.

FIG. 7 is a diagram for describing a method of extracting related attribute factors.

FIG. 8 is a flowchart representing a flow of a series of processing steps of processing circuitry according to the first embodiment.

FIG. 9 is a diagram for describing an outcome estimation method.

FIG. 10 is a diagram showing an example of relationship strength αᵢ for each attribute factor.

FIG. 11 is a diagram showing another example of the relationship strength αᵢ for each attribute factor.

FIG. 12 is a diagram for comparing an attribute factor group of a case where pre-habilitation is performed before treatment with an attribute factor group of a case where pre-habilitation is not performed before treatment.

FIG. 13 is a diagram for comparing an attribute factor group of a case where pre-habilitation is performed before treatment with an attribute factor group of a case where pre-habilitation is not performed before treatment.

FIG. 14 is a diagram showing an example of a configuration of a medical information processing device in a second embodiment.

FIG. 15 is a flowchart showing a flow of a series of processing steps of processing circuitry according to the second embodiment.

FIG. 16 is a diagram showing a medical examination and treatment tree in guidelines.

FIG. 17 is a diagram showing an example in which related attribute factors are displayed.

FIG. 18 is a diagram showing an example in which a certain number of high-level related attribute factors are displayed.

FIG. 19 is a diagram showing an example in which a treatment method is displayed.

FIG. 20 is a diagram showing an example in which an overall map is displayed.

FIG. 21 is a diagram showing an example in which an overall map is displayed.

FIG. 22 is a flowchart showing a flow of a series of processing steps of processing circuitry according to a third embodiment.

FIG. 23 is a diagram for describing a stratification process.

FIG. 24 is a diagram for describing an attribute distribution comparison process.

### DETAILED DESCRIPTION

Hereinafter, a medical information processing device, a medical information processing method, and a storage medium of embodiments will be described with reference to the drawing.

### FIRST EMBODIMENT

A medical information processing device of a first embodiment includes processing circuitry. The processing circuitry acquires a first attribute factor group which is a plurality of attribute factors relating to a disease of a target patient and which is a plurality of attribute factors at a first timing. The processing circuitry estimates a second attribute factor group which is a plurality of attribute factors at a second timing after the first timing on the basis of the first attribute factor group. The processing circuitry outputs information including the first attribute factor group and the second attribute factor group via an output interface. Thereby, a more suitable treatment method can be selected for the patient and his or her family member.

### Configuration of Medical Information Processing System

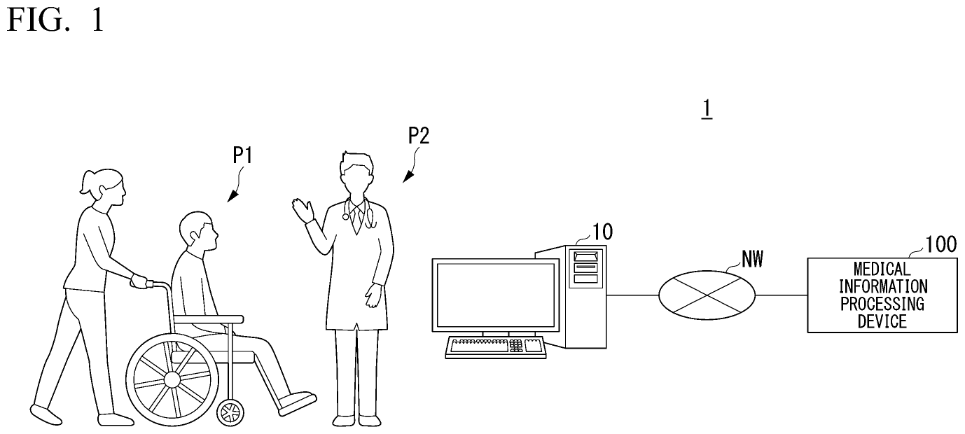

FIG. 1 shows an example of a configuration of a medical information processing system 1 in the first embodiment. The medical information processing system 1 includes, for example, a terminal device 10 and a medical information processing device 100. The terminal device 10 and the medical information processing device 100 are communicatively connected via a communication network NW.

The communication network NW may be the entire information communication network using the telecommunications technology. For example, the communication network NW includes a telephone communication network, an optical fiber communication network, a cable communication network, a satellite communication network, and the like in addition to a wireless/wired local area network (LAN) such as a hospital-based LAN and an Internet network.

The terminal device 10 is a terminal device such as a personal computer, a tablet terminal, and a portable telephone used by a medical worker P2. The medical worker P2 is typically a doctor, but may also be a nurse or another person involved in medical care, or a person involved in community care services. The medical worker P2 inputs, for example, information about a patient who is a treatment target (hereinafter referred to as a target patient P1), to the terminal device 10.

Also, the target patient P1 and his/her family members may input information about the target patient P1 to the terminal device 10 instead of the input by the medical worker P2. Also, the family member may input information about the family member to the terminal device 10 like the target patient P1.

The “treatment” according to the present embodiment may include all medical actions that can be performed before, during, or after the treatment as well as direct treatments such as surgery, drug therapy, chemotherapy, and photoimmunotherapy. For example, the “treatment” of the present embodiment may include “pre-habilitation” performed prior to direct treatment such as surgery, drug therapy, chemotherapy, or photoimmunotherapy, and may include “rehabilitation” performed after the direct treatment.

The terminal device 10 transmits information input by the medical worker P2 or the like to a medical information processing device 100 or receives information from the medical information processing device 100 through the communication network NW.

The medical information processing device 100 receives information from the terminal device 10 via the communication network NW and processes the received information. The medical information processing device 100 transmits the processed information to the terminal device 10 via the communication network NW.

The medical information processing device 100 may be a single device or a system in which a plurality of devices connected via the communication network NW operate in cooperation with each other. That is, the medical information processing device 100 may be implemented by a plurality of computers (processors) included in a distributed computing system or a cloud computing system. Also, the medical information processing device 100 does not necessarily have to be a separate device different from the terminal device 10, but may be a device integrated with the terminal device 10.

### Configuration of Terminal Device

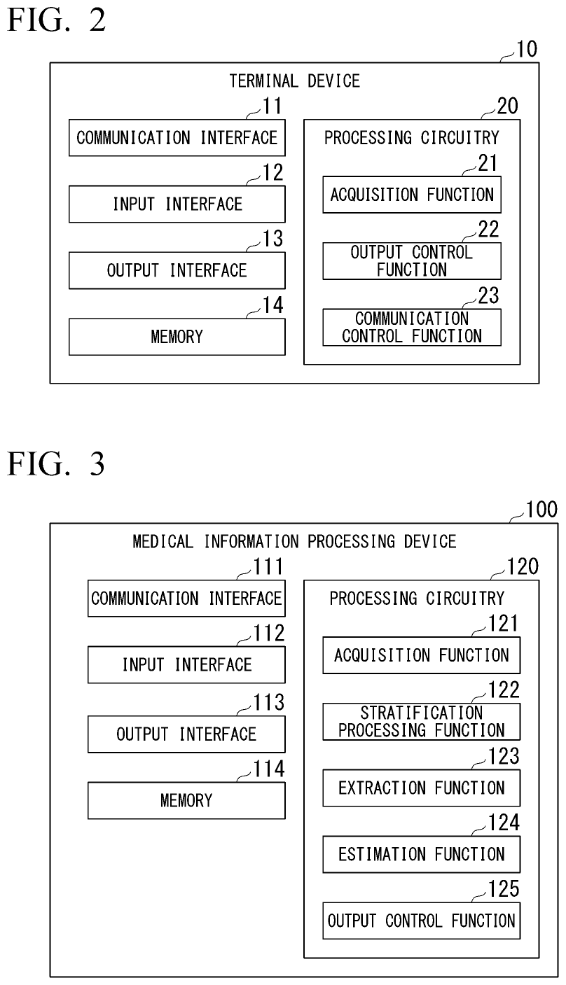

FIG. 2 shows an example of a configuration of the terminal device 10 in the first embodiment. The terminal device 10 includes, for example, a communication interface 11, an input interface 12, an output interface 13, a memory 14, and processing circuitry 20.

The communication interface 11 communicates with the medical information processing device 100 or the like through the communication network NW. The communication interface 11 includes, for example, a network interface card (NIC), an antenna for wireless communication, and the like.

The input interface 12 receives various types of input operations from an operator (for example, a medical worker P2), converts the received input operations into electrical signals, and outputs the electrical signals to the processing circuitry 20. For example, the input interface 12 includes a mouse, a keyboard, a trackball, a switch, a button, a joystick, a touch panel, or the like. The input interface 12 may be, for example, a user interface for receiving a voice input such as a microphone. When the input interface 12 is a touch panel, the input interface 12 may also have a display function of a display 13a to be described below.

Also, in the present specification, the input interface 12 is not limited to those including physical operation components such as a mouse and a keyboard. For example, electrical signal processing circuitry for receiving an electrical signal corresponding to an input operation from an external input device provided separately from the device and outputting the electrical signal to control circuitry is included in the example of the input interface 12.

The output interface 13 outputs information under the control of the processing circuitry 20. For example, the output interface 13 includes the display 13a, a speaker 13b, and the like.

The display 13a displays various types of information. For example, the display 13a displays an image generated by the processing circuitry 20, a graphical user interface (GUI) for receiving various types of input operations from an operator, and the like. For example, the display 13a is a liquid crystal display (LCD), a cathode ray tube (CRT) display, an organic electro luminescence (EL) display, or the like.

The speaker 13b converts various types of information into sounds and outputs the sounds. For example, the speaker 13b outputs information input from the processing circuitry 20 as a sound.

The memory 14 is implemented by a semiconductor memory element such as a random access memory (RAM) or a flash memory, a hard disk, and an optical disk. These non-transitory storage media may be implemented by other storage devices connected via the communication network NW such as a network attached storage (NAS) or an external storage server device. Also, the memory 14 may include a non-transitory storage medium such as a read only memory (ROM) or a register.

The processing circuitry 20 includes, for example, an acquisition function 21, an output control function 22, and a communication control function 23. In the processing circuitry 20, for example, a hardware processor (a computer) executes a program stored in the memory 14 (storage circuit) to implement these functions.

The hardware processor in the processing circuitry 20 is, for example, circuitry such as a central processing unit (CPU), a graphics processing unit (GPU), an application-specific integrated circuit (ASIC), or a programmable logic device (for example, a simple programmable logic device (SPLD), a composite programmable logic device (CPLD), or a field programmable gate array (FPGA)). Instead of storing the program in the memory 14, the program may be directly incorporated in the circuitry of the hardware processor. In this case, the hardware processor reads and executes a program incorporated in the circuitry to implement the function. The program may be previously stored in the memory 14 or stored in a non-transitory storage medium such as a DVD or a CD-ROM, and installed from the non-transitory storage medium to the memory 14 when the non-transitory storage medium is loaded to a drive device (not shown) of the terminal device 10. The hardware processor is not limited to the configuration of single circuitry and may be configured as a single hardware processor by combining a plurality of pieces of independent circuitry to implement each function. Also, a plurality of components may be integrated into one hardware processor to implement each function.

The acquisition function 21 is performed to acquire input information through the input interface 12 or acquire information from the medical information processing device 100 through the communication interface 11.

The output control function 22 is performed to cause the display 13a to display the information acquired by the acquisition function 21 as an image or to cause the information to be output as a sound from the speaker 13b.

The communication control function 23 is performed to transmit information input to the input interface 12 to the medical information processing device 100 via the communication interface 11.

### Configuration of Medical Information Processing Device

FIG. 3 shows an example of a configuration of the medical information processing device 100 in the first embodiment. The medical information processing device 100 includes, for example, a communication interface 111, an input interface 112, an output interface 113, a memory 114, and processing circuitry 120.

The communication interface 111 communicates with the terminal device 10 or the like via the communication network NW. The communication interface 111 includes, for example, a NIC and the like.

The input interface 112 receives various types of input operations from an operator, converts the received input operations into electrical signals, and outputs the electrical signals to the processing circuitry 120. For example, the input interface 112 includes a mouse, a keyboard, a trackball, a switch, a button, a joystick, a touch panel or the like. The input interface 112 may be, for example, a user interface for receiving voice input such as a microphone. When the input interface 112 is a touch panel, the input interface 112 may also have a display function of the display 113a to be described below.

Also, in the present specification, the input interface 112 is not limited to a configuration including physical operation components such as a mouse and a keyboard. For example, electrical signal processing circuitry for receiving an electrical signal corresponding to an input operation from an external input device provided separately from the device and outputting the electrical signal to a control circuit is included in an example of the input interface 112.

The output interface 113 outputs information under control of the processing circuitry 120. For example, the output interface 113 includes the display 113a, a speaker 113b, and the like.

The display 113a displays various types of information. For example, the display 13a displays an image generated by the processing circuitry 120, a GUI for receiving various types of input operations from an operator, and the like. For example, the display 113a is an LCD, a CRT display, an organic EL display, or the like.

The speaker 113b converts various types of information into sounds and outputs the sounds. For example, the speaker 113b outputs information input from the processing circuitry 120 as a sound.

The memory 114 is implemented by a semiconductor memory element such as a RAM, a flash memory, a hard disk, and an optical disk. These non-transitory storage media may be implemented by other storage devices connected via the communication network NW such as an NAS or an external storage server device. Also, the memory 114 may include a non-transitory storage medium such as a ROM or a register.

The processing circuitry 120 includes, for example, an acquisition function 121, a stratification processing function 122, an extraction function 123, an estimation function 124, and an output control function 125. The acquisition function 121 is an example of an “acquisition unit,” the stratification processing function 122 is an example of a “stratification processing unit,” the extraction function 123 is an example of an “extraction unit,” the estimation function 124 is an example of an “estimation unit,” and the output control function 125 is an example of an “output control unit.”

The processing circuitry 120 implements these functions, for example, by executing a program stored in the memory 114 (storage circuitry) with a hardware processor (computer).

The hardware processor in the processing circuitry 120 is, for example, circuitry such as a CPU, a GPU, an application-specific integrated circuit, or a programmable logic device (for example, a simple programmable logic device, a complex programmable logic device, or a field programmable gate array). Instead of storing the program in the memory 114, the program may be directly incorporated in the circuitry of the hardware processor. In this case, the hardware processor reads and executes a program incorporated in the circuitry to implement the function. The program may be previously stored in the memory 114 or stored in a non-transitory storage medium such as a DVD or a CD-ROM, and installed from the non-transitory storage medium to the memory 114 when the non-transitory storage medium is loaded to a drive device (not shown) of the medical information processing device 100. The hardware processor is not limited to the configuration of single circuitry and may be configured as a single hardware processor by combining a plurality of pieces of independent circuitry to implement each function. Also, a plurality of components may be integrated into one hardware processor to implement each function.

### Processing Flow and Pre-Processing for Medical Information Processing Device

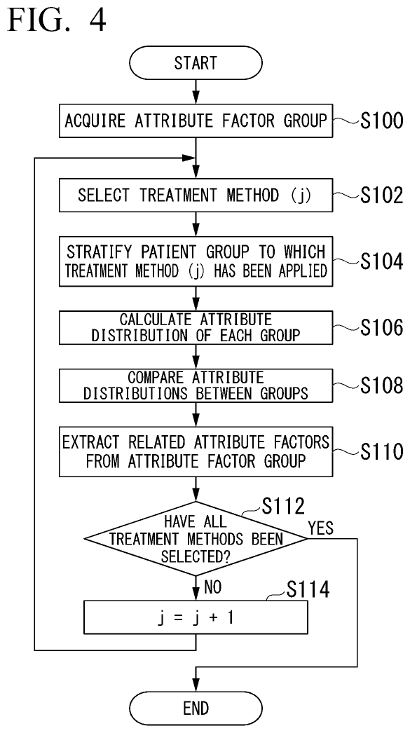

The processing of each function by the processing circuitry 120 of the medical information processing device 100 will be described with reference to the flowchart. FIG. 4 is a flowchart showing a flow of a series of processing steps of the processing circuitry 120 according to the first embodiment. The process of the present flowchart is a preliminary process of an outcome estimation process to be described below. For example, a process of the present flowchart may be performed on a day of absence from medical institutions or at night when the number of outpatients is relatively small.

First, the acquisition function 121 is performed to acquire an attribute factor group consisting of a plurality of attribute factors of different types (step S100).

Attribute factors (attribute parameters) are various factors such as epidemiological information, a patient life function index, a main disease state, a general body state, genetics, a past medical history, a family history, a treatment history, a lifestyle, a diseased organ state, states other than the diseased organ state, and a tumor state. The states other than the diseased organ state are, for example, factors relating to the body's immune function and cardiopulmonary function.

More specifically, the attribute factor may be a factor indicating a current state of the patient, a factor indicating the state transition from the arrival of a patient at a hospital, a factor indicating the state transition during a process of following the patient, a factor indicating anamnesis, or a factor indicating genetic information. Some of a plurality of attribute factors may be omitted or may be replaced with another attribute factor (not illustrated). For example, the attribute factor may include a treatment method, a treatment history, or the like.

The current state includes, for example, an age, a sex, a weight, a blood type, vital signs, coexisting illnesses, and predicted complications. The state transition after the patient's visit includes, for example, a body weight, a cardiac function state, a respiratory state, a metabolic state, image parameters indicating disease characteristics, non-image parameters indicating disease characteristics, and the like. The state transition during a process of following the patient includes, for example, a body weight, a cardiac function state, a respiratory state, a metabolic state, image parameters indicating disease characteristics, non-image parameters indicating disease characteristics, and the like.

For example, it is assumed that the medical worker P2 hears about current states, state transitions, past medical histories, genetic information, and the like of many and unspecified patients from the patients and their families and inputs hearing results to the terminal device 10. In this case, the terminal device 10 transmits the input information as an attribute factor group to the medical information processing device 100. When the communication interface 111 has received the attribute factor group from the terminal device 10, the acquisition function 121 of the medical information processing device 100 is performed to acquire the attribute factor group from the communication interface 111.

The attribute factor group has different types and numbers of attribute factors in accordance with diseases and their treatment methods. For example, the attribute factor group relating to the medical examination and treatment of “cancer” includes 60 to 80 attribute factors. In the process of the present flowchart corresponding to the prior processing, attribute factors highly relevant to the disease are extracted from an attribute factor group including several tens of attribute factors. This attribute factor extraction process will be described below.

Attribute factor degrees (factor values) may be graded in accordance with guidelines established by administrative agencies or the like. Also, the attribute factor degrees may be graded in accordance with diagnosis criteria and a diagnosis tree uniquely determined by each medical institution. Further, the attribute factor degrees may be graded using a database, machine learning, deep learning, or the like.

Next, the stratification processing function 122 is performed to select one treatment method (j) (step S102). j denotes a temporary internal parameter in the processing circuitry 120 and denotes a so-called temporary parameter.

For example, the output control function 22 of the terminal device 10 is performed to cause the display 13a to display a plurality of treatment methods applicable to the patient. The medical worker P2 selects any one of the treatment methods displayed on the display 13a and inputs a selection result to the input interface 12. The communication control function 23 is performed to transmit the selection result of the treatment method input to the input interface 12 by the medical worker P2 to the medical information processing device 100 through the communication interface 11. Accordingly, the stratification processing function 122 of the medical information processing device 100 is performed to acquire a result of selecting the treatment method selected by the medical worker P2 from the terminal device 10 via the communication interface 111.

Subsequently, the stratification processing function 122 is performed to extract a group of patients who were treated in the past using a treatment method (j) from a population containing various patients as a sample and stratifies a patient group which is the sample into a plurality of groups (step S104). The population is an example of “another patient group.”

The population may include, for example, a plurality of patients who were treated in the past under the medical worker P2 who is trying to treat the target patient P1 or under a medical institution where the medical worker P2 works. Medical institutions include hospitals, clinics, and other facilities where medical care is provided. Also, the population may be a patient population in medical statistics. On the basis of outcomes, the population can be stratified into a plurality of groups (also called classification, grouping, or clustering).

Outcomes indicate various index values that a patient must achieve in treating the patient, and include, for example, time taken to remission, a survival period, a length of life for self-reliance, and the like.

For example, if the treatment method (j) acquired in the processing of the S102 is “AAA,” the stratification processing function 122 is performed to extract a plurality of patients who were treated in a treatment method “AAA” in the past from the population as a sample.

The stratification processing function 122 is performed to stratify a plurality of patients (i.e., samples) extracted from the population on the basis of the treatment method (j) into a plurality of groups. The stratification processing function 122 is performed to select one or more index values (typically select one index value) from among a plurality of index values included in an outcome before sample stratification.

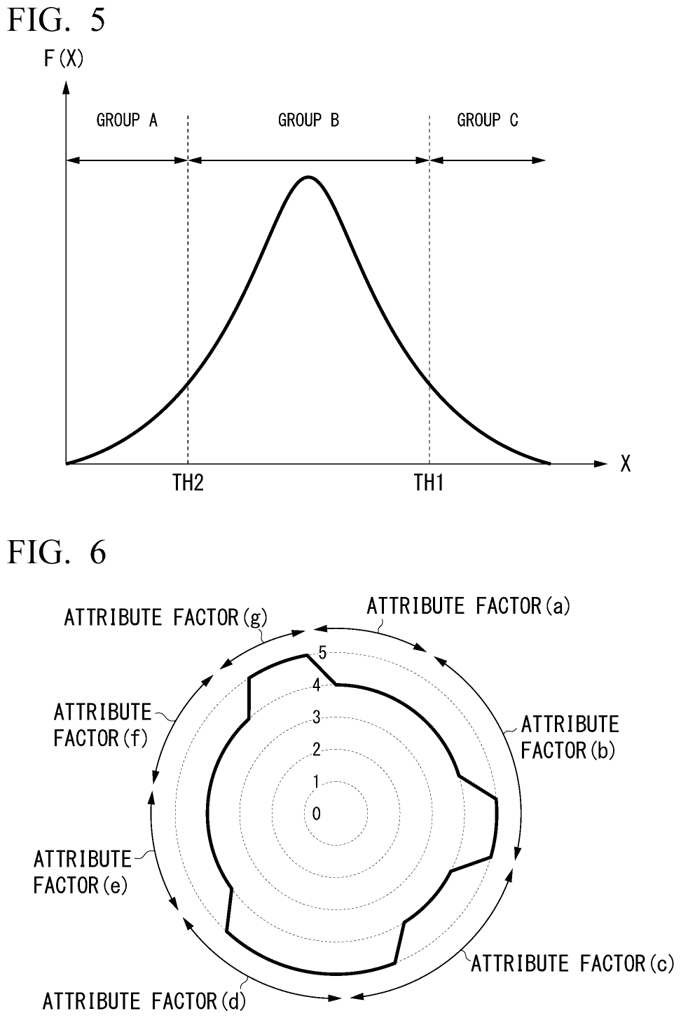

FIG. 5 is a diagram for describing the stratification process. As shown, for example, the stratification processing function 122 is performed to calculate a probability density distribution F(X) of samples when the outcome is set as a probability variable X. The stratification processing function 122 is performed to stratify the samples into a plurality of groups in accordance with a certain criterion on the probability density distribution F(X).

For example, the stratification processing function 122 may be performed to classify a group in which the probability variable X is less than a second threshold value TH2 as group A, classify a group in which the probability variable X is greater than or equal to the second threshold value TH2 and less than the first threshold value TH1 as group B, and classify a group in which the probability variable X is greater than or equal to the first threshold value TH1 as group C. The first threshold value TH1 and the second threshold value TH2 may be, for example, a fixed value decided on the basis of a medical statistical result or guideline, or may be a reference value to or from which a predetermined margin is added or subtracted with respect to the national average, the average in each medical institution, or the like. Each of the first threshold value TH1 and the second threshold value TH2 may have a standard deviation such as ±1σ, ±2σ, or ±3σ. The number of threshold values may be not limited to two and may be one or three or more. That is, the number of groups may be two or four or more.

For example, when the treatment method (j) is a “cancer treatment method” and the outcome is a “survival period,” the stratification processing function 122 is performed to extract a patient (cancer patient) to which the cancer treatment method has been applied as a sample from the population in a filtering process and calculate the survival period of the extracted sample, i.e., the probability density distribution F(X) for which the survival period of the cancer patient is a probability variable X. The stratification processing function 122 is performed to stratify samples into, for example, three groups A, B, and C, on the probability density distribution F(X) relating to the survival period of the cancer patient. In this case, group A becomes a group of cancer patients having a longer survival period, group B becomes a group having a shorter survival period than group A of cancer patients, and group C becomes a group having a shorter survival period than group B of cancer patients. That is, group A is a group having the most improved outcome after treatment is applied thereto, group B is a group having the second most improved outcome after group A, and group C is a group having the least improved outcome. Group C is an example of a “first group” and group A is an example of a “second group.”

Returning to the flowchart description, the stratification processing function 122 is subsequently performed to calculate a distribution (hereinafter referred to as an attribute distribution) quantitatively expressing the attribute factor of each group (step S106).

FIG. 6 is a diagram showing an example of an attribute distribution. As shown, for example, the attribute distribution may be represented as a radar chart in which the degree of each attribute factor is graded in five steps from 0 to 5. That is, the values of the attribute factors may be expressed as a distribution like a radar chart after being normalized so that the minimum value becomes 0 and the maximum value becomes 5. In region (a) of the attribute distribution, for example, attribute factors relating to the age, the sex, and the blood type are plotted. In region (b), for example, attribute factors relating to the general body state are plotted. In region (c), for example, attribute factors relating to the lifestyle are plotted. In region (d), for example, attribute factors relating to a past medical history and genetics are plotted. In region (e), for example, attribute factors relating to states other than a diseased organ state are plotted. In region (f), for example, attribute factors relating to the diseased organ state are plotted. In region (g), for example, attribute factors relating to the tumor state are plotted.

For example, the stratification processing function 122 is performed to average attribute distributions of a plurality of patients (samples) included in each group and uses the average of the attribute distributions as the attribute distribution of each group. Specifically, when group A includes 100 patients, the stratification processing function 122 is performed to average the attribute distributions of the 100 patients and set one attribute distribution obtained by averaging the attribute distributions of the 100 patients as the attribute distribution of group A. The stratification processing function 122 may be performed to calculate an attribute distribution of each group by similarly averaging attribute distributions of a plurality of patients with respect to other groups such as group B and group C. The attribute distribution of group C is an example of a “first attribute distribution” and the attribute distribution of group A is an example of a “second attribute distribution.”

The attribute factors (a general body state, a lifestyle, states other than a diseased organ state, and a diseased organ state) in (b), (c), (e), and (f) are controllable attribute factors (i.e., control factors) before, after, or in a process in which direct treatment such as surgery is applied. On the other hand, attributes (an age, a sex, a blood type, a past medical history, genetics, and a tumor state) of (a), (d), and (g) are attribute factors (i.e., non-control factors) that cannot be controlled before, after, or in the process in which direct treatment such as surgery is applied to a patient.

Although a case where the attribute distribution is a radar chart has been described in the example in FIG. 6, the present invention is not limited thereto. For example, the attribute distribution may be represented by other statistical diagrams, such as histograms, stacked graphs, and heat maps. Also, the number of steps of the attribute factor is not limited to 5. For example, a certain criterion may be set and the number of steps of the attribute factor may be 4 or less or 6 or more. The criterion may be a fixed value obtained with reference to medical statistics and guidelines, may be set as a reference value (a national average or an institutional average)±a set margin, and may be in or out of the range of ±2σ or 80% CV for the overall distribution. Non-quantified information (a medical history, a family history, and a lifestyle) can be scored by a degree of influence on a disease.

Also, the stratification processing function 122 may be performed to calculate one index value (scalar value) by integrating all attribute factors of the groups instead of or in addition to a process of calculating the attribute factors of the groups as a distribution. For example, if the number of attribute factors is defined as n, the stratification processing function 122 may be performed to use a sum T(i)=Σαᵢ×τ(i), which takes into account the degree of influence αᵢ on the outcome of each attribute factor (τ(i); i=1 to n), as an index value indicating all the attribute factors. Also, when attribute information factors are grouped according to each category, fₙ(i)=Σαᵢ×τ(i) can be expressed in a similar method. For example, f₁₋₆(i)=Σαᵢ×τ(i) can be expressed when the tumor state (f₁), the entire diseased organ (f₂), the general body state (living aspect) (f₃), the states other than the diseased organ state (an immune function (f₄), a cardiac function (f₅), and a respiratory function (f₆)) are combined.

Returning to the flowchart description, the extraction function 123 is subsequently performed to compare attribute distributions of groups (step S108) and extract one or more related attribute factors from the attribute factor group on the basis of a comparison result (step S110). A related attribute factor is the attribute factor highly relevant to a disease as described above and is specifically an attribute factor having a higher degree of influence on (1) the selection of a disease treatment method and/or (2) the improvement of an outcome than the other attribute factors.

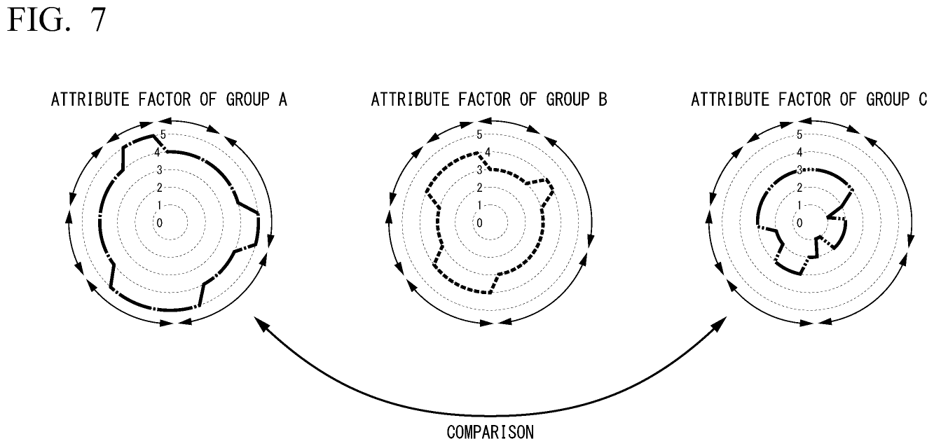

FIG. 7 is a diagram for describing a method of extracting the related attribute factor. As shown, for example, the extraction function 123 is performed to compare the attribute distribution of group A having the best outcome with the attribute distribution of group C having the worst outcome among three groups. For example, the extraction function 123 may be performed to extract, as a related attribute factor, an attribute factor having a variation amount equal to or greater than a threshold value when the attribute distribution of group A and the attribute distribution of group C are compared.

Also, the extraction function 123 may be performed to extract an attribute factor for which relationship strength αᵢ is equal to or greater than a threshold value as a related attribute factor. The relationship strength αᵢ is an intensity of a degree of influence of each attribute factor fᵢ on (1) the selection of a disease treatment method and/or (2) the improvement of an outcome. For example, the relationship strength αᵢ is obtained according to a correlation coefficient in statistical analysis, a distribution probability of each classifier for each random forest layer, and output information from machine learning.

Returning to the flowchart description, the stratification processing function 122 is performed to determine whether or not all treatment methods have been selected (step S112). The stratification processing function 122 ends the process of the present flowchart when all the treatment methods have been selected.

On the other hand, when all the treatment methods have not yet been selected, the stratification processing function 122 is performed to increment a temporary parameter j (step S114) and returns the process to the S102. That is, the stratification processing function 122 is performed to reselect a treatment method that has not yet been selected as a new treatment method (j). Thereby, it is possible to stratify the population into a plurality of groups for each treatment method and it is further possible to extract related attribute factors appropriate for each treatment method.

### Processing Flow Outcome Estimation Process of Medical Information Processing Device

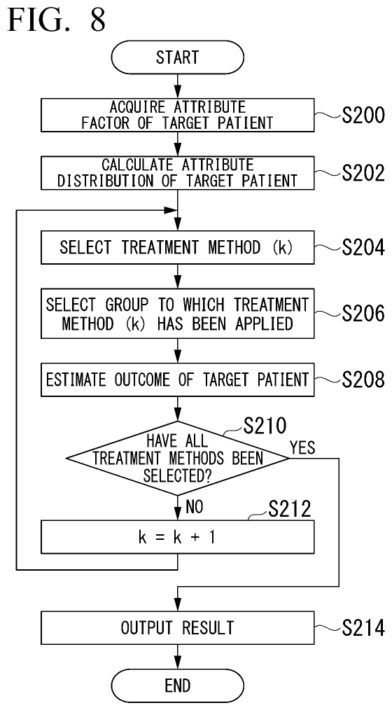

A process of each function by the processing circuitry 120 of the medical information processing device 100 will be described with reference to the flowchart. FIG. 8 is a flowchart showing a flow of a series of processing steps of the processing circuitry 120 according to the first embodiment. For example, the process of the present flowchart is executed before direct treatment such as surgery, drug therapy, chemotherapy, and photoimmunotherapy is performed on the target patient P1.

First, the acquisition function 121 is performed to acquire a plurality of attribute factors (hereinafter referred to as an attribute factor group) relating to the disease of the target patient P1 before a direct treatment such as a surgical operation or a drug therapy is performed (step S300). The attribute factor group includes at least the related attribute factors, and more preferably, the attribute factor group may be limited to only the related attribute factors.

Subsequently, the stratification processing function 122 is performed to calculate an attribute distribution quantitatively expressing the attribute factor group of the target patient P1 (step S302). The attribute distribution of the target patient P1 is an example of a “third attribute distribution.”

Subsequently, the stratification processing function 122 is performed to select one treatment method (k) applicable to the target patient P1 (step S304). k is a temporary internal parameter in the processing circuitry 120 like j, i.e., a so-called temporary parameter.

Subsequently, the estimation function 124 is performed to select a group to which the treatment method (k) has been applied from among a plurality of groups that have been stratified for each treatment method in pre-processing (step S306). In other words, the estimation function 124 is performed to select, from among a plurality of stratified groups, a group that suffers from the same disease as the target patient P1 and to which a treatment method identical to the treatment method (k) to be applied to the target patient P1 was applied in the past.

Subsequently, the estimation function 124 is performed to estimate the outcome of the target patient P1 at a future time t on the basis of the attribute factor of the group to which the treatment method (k) was applied (step S308).

Subsequently, the estimation function 124 is performed to determine whether or not all the treatment methods applicable to the target patient P1 have been selected (step S310).

When all the treatment methods have not yet been selected, the estimation function 124 is performed to increment the temporary parameter k (step S312) and returns the process to S204. That is, the estimation function 124 is performed to reselect a treatment method that has not yet been selected as a new treatment method (k).

When all the treatment methods have been selected, the output control function 125 is performed to output a calculation result or the like based on the estimation function 124 via the output interface 113 (step S314).

For example, the output control function 125 may be performed to cause the display 113a of the output interface 113 to display information (hereinafter referred to as output information) including the attribute factor group of the target patient P1 acquired in the processing of the S200, the attribute factor group of the target patient P1 estimated in the estimation function 124, the outcome of the target patient P1 estimated in the estimation function 124, and the like. Also, the output control function 125 may transmit the output information to the terminal device 10 via the communication interface 111. Thus, the process of the present flowchart ends.

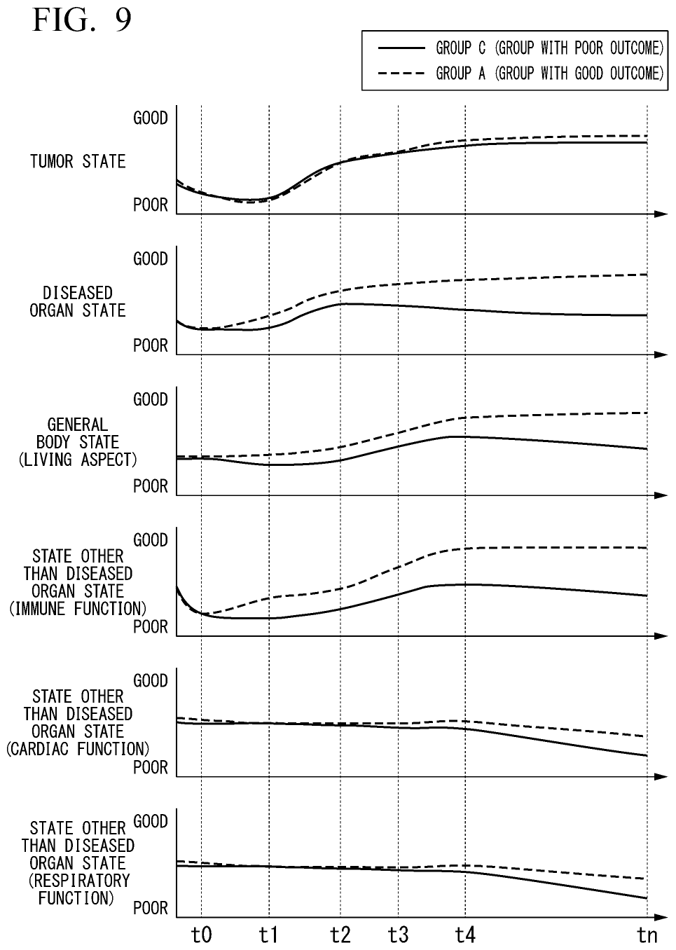

FIG. 9 is a diagram for describing an outcome estimation method. As in an example shown in FIG. 9, an attribute factor group of a group stratified in pre-processing can be divided into, for example, a tumor state, a diseased organ state, a general body state (living aspect), and the states other than the diseased organ state (an immune function, a cardiac function, and a respiratory function). When the attribute factor groups of each group are present at sampling times such as times t0, t1, t2, t3, t4, . . . , tn, the horizontal axis represents time, and the vertical axis represents each attribute factor, a change corresponding to time of each attribute factor can be graphed as shown in FIG. 9. For example, sampling time t0 can be the time of visit, time t1 can be immediately before direct treatment such as a surgical operation, time t2 can be immediately after the direct treatment, time t3 can be 6 months after the treatment, time t4 can be 1 year after the treatment, and time tn can be 5 years after the treatment. Time t0 or time t1 is an example of a “first timing” and each of times t2, t3, t4, tn is an example of a “second timing.”

For example, when the target patient P1 is still in a stage before the direct treatment (i.e., at time t0 or t1), the estimation function 124 is performed to estimate the attribute factor group of the target patient P1 (i.e., an attribute factor group at each of times t2, t3, t4, tn) expected to change after the direct treatment on the basis of temporal changes in the attribute factor groups of the group to which a treatment method identical to that of the target patient P1 has been applied.

More specifically, the estimation function 124 may be performed to estimate an attribute factor group f(tn) of the target patient P1 at a future time tn in accordance with Eq. (1).

f(tn)=z(f₁(t), f₂(t), f₃(t), f₄(t), f₅(t), f₆(t)) (1)

For example, f₁(t) may indicate a tumor state at time t of the target patient P1, f₂(t) may indicate the entire diseased organ at time t of the target patient P1, f₃(t) may indicate a general body state at time t of the target patient P1, f₄(t) may indicate a state other than the diseased organ state (an immune function) at time t of the target patient P1, f₅(t) may indicate a state other than the diseased organ state (a cardiac function) at time t of the target patient P1, and f₆(t) may indicate a state other than the diseased organ state (a respiratory function) at time t of the target patient P1. z is a sixth-order equation having any weight coefficients z₁ to z₆. Eq. (1) can be obtained by statistically analyzing each attribute factor or can be obtained by performing machine learning with the attribute factor and the state of each time as the input.

For example, the estimation function 124 is performed to estimate the attribute factor group of the target patient P1 who changes after treatment (t2, t3, t4, . . . , tn) by inputting the attribute factor group of the target patient P1 at either time (t0) of visit or time (t1) immediately before treatment or both times as an explanatory variable to Eq. (1) and estimate a future outcome of the target patient P1.

A typical example will be described. For example, at time t1 immediately before direct treatment is applied, it is assumed that the target patient P1 wants to know an extent to which an outcome (for example, a survival rate) changes at time t3 that is 6 months after direct treatment is applied. In this case, an estimation function 124 is performed to input each attribute factor at time t3 to the explanatory variables f₁(t) to f₆(t) of Eq. (1) and estimate each attribute factor at time t3. For example, if the attribute factor of the target patient P1 is close to the attribute factor of group A at time t3, it is possible to estimate that the outcome of the target patient P1 can be suitably maintained six months after treatment.

When the outcome of the target patient P1 is estimated, the estimation function 124 may be performed to calculate relationship strength αᵢ that is an intensity of a degree of influence of each attribute factor fᵢ on the outcome improvement or the like and multiply the relationship strength αᵢ by each attribute factor fᵢ(t) that is an explanatory variable of Eq. (1) as described above. That is, the functional state fn(t) at a certain point of time (t) can be expressed as fn(i)=Σαᵢ×τ(i).

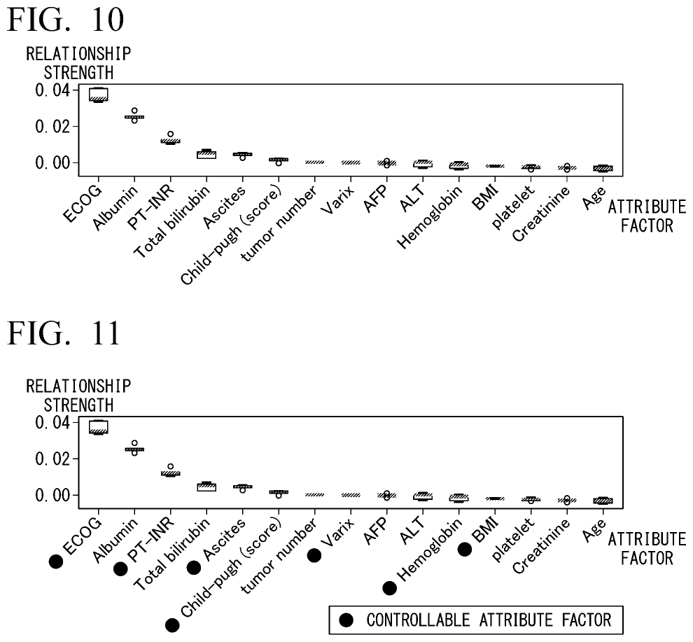

FIG. 10 is a figure showing an example of the relationship strength αᵢ for each attribute factor. As shown, relationship intensities αᵢ of the attribute factors “ECOG,” “Albumin,” and “PT-INR” are greater than those of the other attribute factors. These attribute factors “ECOG,” “Albumin,” and “PT-INR” will be more weighted than other attribute factors.

As described above, the attribute factors of a patient can be classified into control factors such as a general body state, a lifestyle, states other than a diseased organ state, and a diseased organ state and non-control factors such as an age, a sex, a blood type, a past medical history, genetics, and a tumor state. Therefore, the estimation function 124 may be performed to calculate relationship strength αᵢ after classifying a control factor and a non-control factor.

FIG. 11 is a diagram showing another example of relationship strength αᵢ for each attribute factor. The estimation function 124 is performed to obtain an average value and a variance value and obtain a range of ±2σ or 80% CV in terms of a distribution in each of group B having an average outcome, group C having a poor outcome, and group A having a good outcome in terms of control factors. The value may be held and used as a database or may be calculated every time. Also, guidelines or evidence values may be used. The attribute factor group of the target patient P1 may be normalized by these values. The output control function 125 may be performed to output a relationship diagram between an attribute factor and relationship strength αᵢ as illustrated in FIG. 10 and FIG. 11 via the output interface 113.

Also, the functional state fn(t) at a certain point in time t is fn(i)=Σαᵢ×τ(i). This can be expressed as fn(i)=Σαᵢ×τ(i)×θi if the effect of adjustment of the control factor is set to a factor θi in consideration of a control factor and a non-control factor. The control factor is treated as external variable information. The factor θi of the effect of the adjustment of the control factor may be calculated from a rate of a change in the factor per unit period or may be set as a constant. For example, the coefficient θi=1 is used for the non-control factor.

Also, in the case of a new treatment method, some attribute factor groups may not be present in the attribute factor groups of an existing patient population. In such cases, a range in which outcomes are improved is set and used on the basis of evidence from clinical trials and the latest studies relating to new treatment methods.

Also, a relational expression for obtaining a change in the patient attribute information may be updated after being determined for each facility or country. Also, it may be operated only by an execution function for creating a relational expression or it may be used in a fixed condition together with an attribute factor group constituting the relational expression (i.e., an attribute factor that is an explanatory variable in the equation).

Further, the estimation function 124 may be performed to estimate how the outcome of the target patient P1 is improved when control factors included in the attribute factor group of the target patient P1 are adjusted so that the attribute distribution of the target patient P1 calculated in the processing of the S202 approaches the attribute distribution of group A having a good outcome.

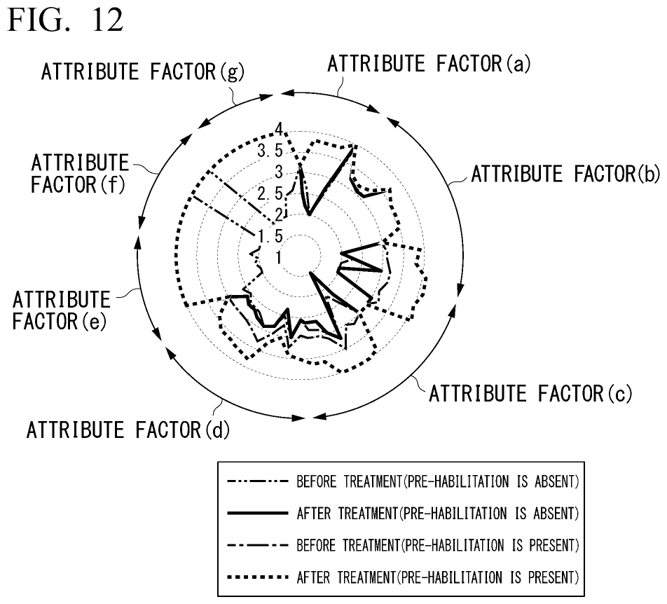

FIG. 12 is a diagram for comparing an attribute factor group of a case where pre-habilitation is performed before treatment with an attribute factor group of a case where pre-habilitation is not performed before treatment. For example, when the target patient P1 has performed pre-habilitation before treatment, control factors such as the general body state and the lifestyle are expected to be improved to better values. More specifically, when exercise has been done as a part of pre-habilitation, it is expected that the body weight of the target patient P1 approaches a standard value (a body weight in group A). In such a case, among the attribute factor groups of the target patient P1 obtained at the time of visit (t0) or immediately before treatment (t1), control factors such as the general body state and the lifestyle are adjusted to be close to the attribute factors such as the general body state and the lifestyle of group A and the attribute factor group (including the adjusted control factors) of the target patient P1 are input as explanatory variables to Eq. (1). Thereby, it is possible to estimate the future outcome of the target patient P1 when pre-habilitation has been performed before treatment.

The output control function 125 may be performed to output a comparison diagram of the attribute distribution shown in FIG. 12 via the output interface 113. Thereby, the target patient P1 or a family member thereof can ascertain how much the post-treatment outcome will be improved according to whether or not pre-habilitation is performed before the treatment.

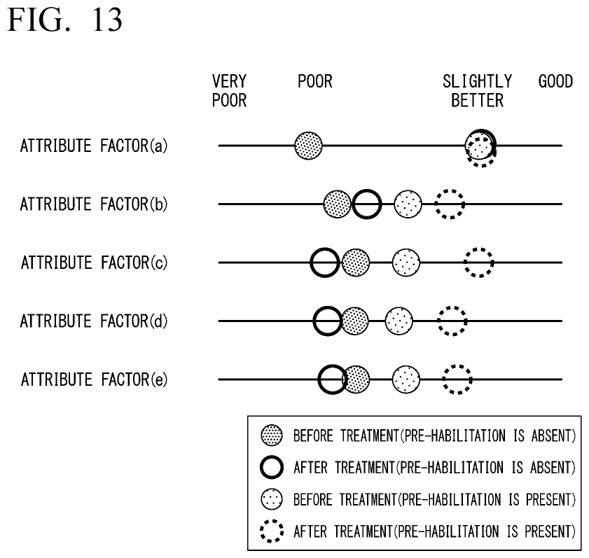

FIG. 13 is a diagram for comparing an attribute factor group of a case where pre-habilitation is performed before treatment with an attribute factor group of a case where pre-habilitation is not performed before treatment. In FIG. 13, “poor” indicates that the attribute factor is close to an attribute factor of group C with a poor outcome and “good” indicates that the attribute factor is close to an attribute factor of group A with a good outcome. The output control function 125 may be performed to output a comparison diagram of the attribute factor group as illustrated in FIG. 13 via the output interface 113.

According to the first embodiment described above, the medical information processing device 100 acquires an attribute factor group (an example of a “first attribute factor group”) of a target patient P1 before treatment is applied. The medical information processing device 100 estimates an attribute factor group (an example of a “second attribute factor group”) of the target patient P1 after the treatment is applied on the basis of the attribute factor group of the target patient P1 before the treatment is applied. Also, the medical information processing device 100 outputs information including an attribute factor group of the target patient P1 before the treatment is applied and an attribute factor group of the target patient P1 after the treatment is applied via the output interface 113. Thereby, a degree of change in the attribute factor, an outcome, or the like after treatment is applied can be presented to a patient and his or her family member. As a result, the patient and his or her family member can select a more suitable treatment method.

### MODIFIED EXAMPLES OF FIRST EMBODIMENT

The modified example (another embodiment) of the first embodiment will be described below. Although a case where the terminal device 10 and the medical information processing device 100 are mutually different devices in the above-described first embodiment has been described, the present invention is not limited thereto. For example, the terminal device 10 and the medical information processing device 100 may be one integrated device. For example, the processing circuitry 20 of the terminal device 10 may further include some or all of the stratification processing function 122, the extraction function 123 and the estimation function 124 provided in the processing circuitry 120 of the medical information processing device 100 in addition to the acquisition function 21, the output control function 22 and the communication control function 23. In this case, the terminal device 10 can perform the above-described processes of various types of flowcharts in a stand-alone (offline) way.

Although a case where processes of the flowcharts shown in FIG. 4 and FIG. 8 are executed only by the medical information processing device 100 has been described, the present invention is not limited thereto. For example, some of the processes of these flowcharts may be executed by the terminal device 10.

### SECOND EMBODIMENT

Hereinafter, a second embodiment will be described. The second embodiment is different from the first embodiment in that related attribute factors which are attribute factors highly relevant to the disease are identified in a group of attribute factors relating to the disease of the target patient P1.

For example, it is said that there are 60 to 80 attribute factors relating to the medical examination and treatment of cancer. These many attribute factors are present in information systems of different medical departments, for example, such as radiology and oncology, and cannot be covered by electronic medical records alone. Therefore, there is a situation where it is not possible to manage all attribute factors. Furthermore, there are a large number of attribute factors and there are various systems capable of retrieving the attribute factors. It takes a long time to examine all the attribute factors and it is easy to overlook them. Also, focusing on selecting the appropriate treatment method may overlook the relationship with patient outcomes.

In the second embodiment, to solve this problem, related attribute factors highly relevant to diseases are identified in a group of attribute factors of a target patient P1 and information based on the related attribute factors is output. Thereby, it is possible to select a treatment method of improving the outcome of a patient while increasing the reliability and efficiency of collecting attribute factors relating to the disease.

The differences from the first embodiment are described mainly and content identical to that of the first embodiment is omitted. Also, in the description of the second embodiment, parts identical to those of the first embodiment are denoted by the same reference signs.

### Configuration of Medical Information Processing Device

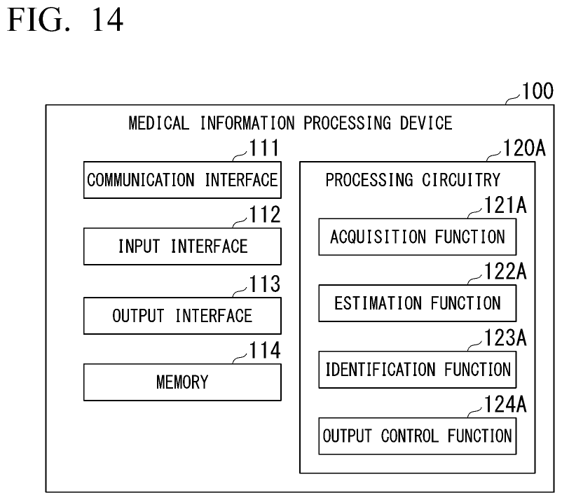

FIG. 14 shows an example of a configuration of a medical information processing device 100 in the second embodiment. The medical information processing device 100 includes, for example, a communication interface 111, an input interface 112, an output interface 113, a memory 114, and processing circuitry 120A.

Because the communication interface 111, the input interface 112, the output interface 113, and the memory 114 are similar to those of the first embodiment, the description thereof is omitted here.

The processing circuitry 120A in the second embodiment includes, for example, an acquisition function 121A, an estimation function 122A, an identification function 123A, and an output control function 124A. The acquisition function 121 is an example of an “acquisition unit,” the estimation function 122A is an example of an “estimation unit,” the identification function 123A is an example of an “identification unit,” and the output control function 124A is an example of an “output control unit.”

As in the first embodiment, the processing circuitry 120 in the second embodiment implements these functions, for example, by executing a program stored in the memory 114 (storage circuitry) with a hardware processor (computer).

As in the first embodiment, the hardware processor in the processing circuitry 120A in the second embodiment is, for example, circuitry such as a CPU, a GPU, an application-specific integrated circuit, or a programmable logic device (for example, a simple programmable logic device, a complex programmable logic device, or a field programmable gate array). Instead of storing the program in the memory 114, the program may be directly incorporated in the circuitry of the hardware processor. In this case, the hardware processor reads and executes a program incorporated in the circuitry to implement the function. The program may be previously stored in the memory 114 or stored in a non-transitory storage medium such as a DVD or a CD-ROM, and installed from the non-transitory storage medium to the memory 114 when the non-transitory storage medium is loaded to a drive device (not shown) of the medical information processing device 100. The hardware processor is not limited to the configuration of single circuitry and may be configured as a single hardware processor by combining a plurality of pieces of independent circuitry to implement each function. Also, a plurality of components may be integrated into one hardware processor to implement each function.

### Processing Flow for Medical Information Processing Device

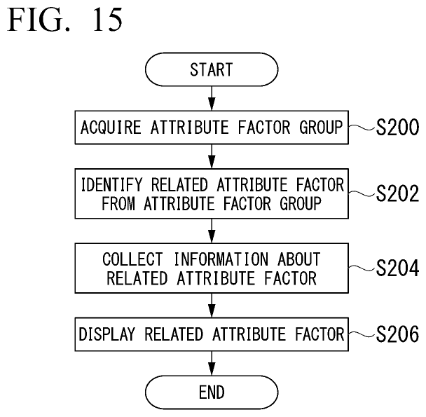

The process of each function of the processing circuitry 120A of the medical information processing device 100 is described with reference to the flowchart. FIG. 15 is a flowchart showing a flow of a series of processing steps of the processing circuitry 120A according to the second embodiment.

First, the acquisition function 121A is performed to acquire a group of attribute factors relating to diseases of a target patient P1 (step S200).

The attribute factor group is composed of a plurality of attribute factors of different types. Attribute factors (attribute parameters) are various factors such as epidemiological information, a patient life function index, a main disease state, a general body state, genetics, a past medical history, a family history, a treatment history, a lifestyle, a diseased organ state, states other than the diseased organ state, and a tumor state. The states other than the diseased organ state are, for example, factors relating to the body's immune function and cardiopulmonary function.

More specifically, the attribute factor may be a factor indicating a current state of the patient, a factor indicating the state transition from the arrival of a patient at a hospital, a factor indicating the state transition during a process of following the patient, a factor indicating anamnesis, or a factor indicating genetic information. Some of a plurality of attribute factors may be omitted or may be replaced with another attribute factor (not illustrated). For example, the attribute factor may include a treatment method, a treatment history, or the like.

The current state includes, for example, an age, a sex, a weight, a blood type, vital signs, coexisting illnesses, and predicted complications. The state transition after the patient's visit includes, for example, a body weight, a cardiac function state, a respiratory state, a metabolic state, image parameters indicating disease characteristics, non-image parameters indicating disease characteristics, and the like. The state transition during a process of following the patient includes, for example, a body weight, a cardiac function state, a respiratory state, a metabolic state, image parameters indicating disease characteristics, non-image parameters indicating disease characteristics, and the like.

For example, it is assumed that the medical worker P2 hears about current states, state transitions, past medical histories, genetic information, and the like of many and unspecified patients from the patients and their families and inputs hearing results to the terminal device 10. In this case, the terminal device 10 transmits the input information as an attribute factor group to the medical information processing device 100. When the communication interface 111 has received the attribute factor group from the terminal device 10, the acquisition function 121A of the medical information processing device 100 is performed to acquire the attribute factor group from the communication interface 111.

The attribute factor group has different types and numbers of attribute factors in accordance with diseases and their treatment methods. For example, the attribute factor group relating to the medical examination and treatment of “cancer” includes 60 to 80 attribute factors. In the process of the present flowchart, attribute factors highly relevant to the disease are identified from an attribute factor group including several tens of attribute factors.

Attribute factor degrees (factor values) may be graded in accordance with a guideline established by an administrative agency or the like. Also, the attribute factor degrees may be graded in accordance with diagnosis criteria and a diagnosis tree determined by each medical institution. Further, the attribute factor degrees may be graded using a database, machine learning, deep learning, or the like. Non-quantified attribute factors (for example, a medical history, a family history, a lifestyle, and the like) can be quantified by the degree of influence on the disease.

Subsequently, the identification function 123A is performed to identify one or more related attribute factors (typically, a plurality of related attribute factors) in the attribute factor group of the target patient P1 (step S202). A related attribute factor is an attribute factor highly relevant to a disease as described above and is specifically an attribute factor having a higher degree of influence on at least one or both of (1) the selection of a disease treatment method and (2) the improvement of an outcome than the other attribute factors.

Outcomes indicate various index values that a patient must achieve in treating the patient, and include, for example, time taken to remission, a survival period, a length of life for self-reliance, and the like.

For example, the identification function 123A may be performed to refer to the guideline or the latest evidence and identify a reference attribute factor determined in the guideline or the latest evidence as the related attribute factor. More specifically, the identification function 123A is performed to compare each attribute factor included in the attribute factor group of the target patient P1 with a reference attribute factor determined by the guideline or the latest evidence and identifies, as the related attribute factor, an attribute factor in which the deviation from the reference attribute factor is greater than or equal to a threshold value in the attribute factor group.

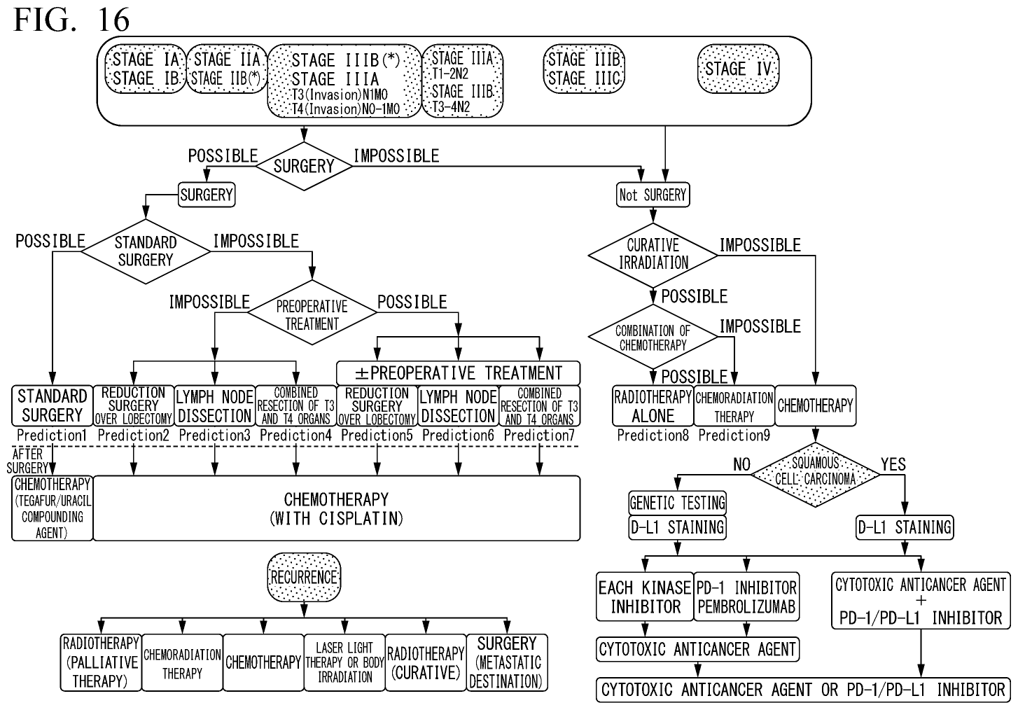

Also, for example, the identification function 123A may be performed to use a medical examination and treatment tree of the guideline as shown in FIG. 16 to find an attribute factor having a higher degree of influence on the selection of a disease treatment method than other attribute factors by designating the attribute factor and the treatment method selection as a relationship between input and output and identify the found attribute factor as the related attribute factor.

Also, for example, the identification function 123A may be performed to use statistical analysis and machine learning to find an attribute factor having a higher degree of influence on the outcome than other attribute factors by designating the attribute factor and the outcome as a relationship between input and output and identify the found attribute factor as the related attribute factor. For example, the identification function 123A is performed to compare each attribute factor included in the attribute factor group of the target patient P1 with an attribute factor of another patient group with an improved outcome and identifies, as the related attribute factor, an attribute factor in which the deviation from the attribute factor of the other patient group is greater than or equal to a threshold value in the attribute factor group. The attribute factor of the other patient group with the improved outcome is an example of a “second attribute factor.”

Subsequently, the identification function 123A is performed to collect information about the related attribute factor (step S204). For example, the identification function 123A is performed to access the terminal device 10 which can be used by the medical worker P2 and a system in a medical institution via the communication interface 111 and collect various information such as text and a numerical value associated with the related attribute factor from medical data of the target patient P1 using the name of the related attribute factor as a keyword. The medical data of the target patient P1 may be electronic data of a general-purpose format such as Portable Document Format or electronic data of a dedicated format or a dedicated protocol (for example, HL7 V2.5 or HL7 FHIR) determined between the systems.

Subsequently, the output control function 124A is performed to output the collected information about the related attribute factor (step S206). For example, the output control function 124A may be performed to cause the display 113a of the output interface 113 to display the information about the related attribute factor. Also, the output control function 124A may also be performed to transmit information about the related attribute factor to the terminal device 10 via the communication interface 111. Thereby, the process of the present flowchart ends.

### DISPLAY EXAMPLE 1

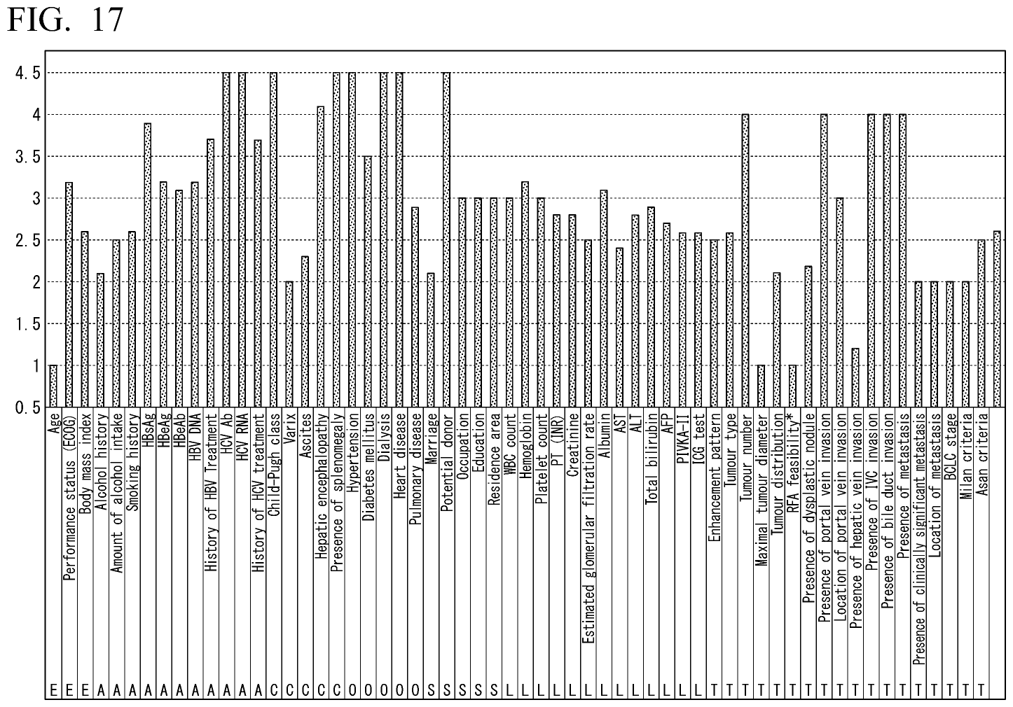

FIG. 17 is a diagram showing an example in which a related attribute factor is displayed. As shown, the output control function 124A may be performed to cause the display 113a to display a plurality of related attribute factors as a list. At this time, the output control function 124A may be performed to set a certain reference and normalize the scale of each of the plurality of related attribute factors to three steps (within a reference range, above the reference range, and below the reference range) or five steps. The reference may be a fixed value obtained with reference to medical statistics and guidelines, may be set with a positive or negative margin for the reference value (a national average or an average within a facility), or may be in or out of a range of 2σ or 80% CV in the overall distribution.

When a plurality of related attribute factors are displayed, the output control function 124A may display only a prescribed number of high-level related attribute factors having a high degree of influence on the selection of the disease treatment method among a plurality of related attribute factors or may display only a prescribed number of high-level related attribute factors having a high degree of influence on the improvement of the outcome.

### DISPLAY EXAMPLE 2

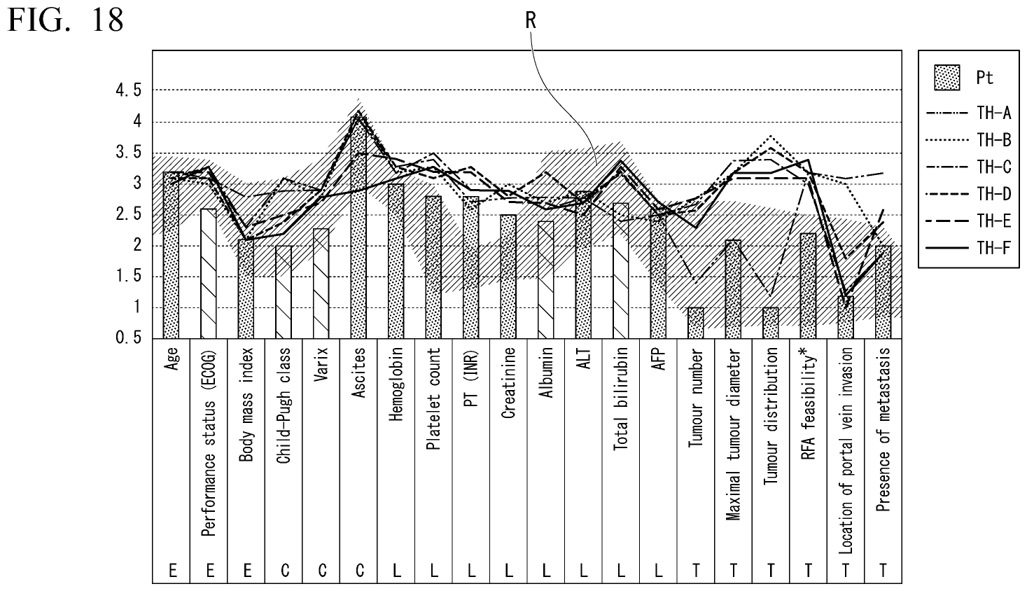

FIG. 18 shows an example in which a predetermined number of high-level related attribute factors are displayed. As shown, the output control function 124A may be performed to cause the display 113a to display only a predetermined number of high-level related attribute factors as a list. At this time, the output control function 124A may be performed to execute a display process together with an optimal range R for application to each treatment method. The optimal range R is set to be within a range of ±2σ or 80% CV when the average value and normal distribution of each of the related attribute factors are assumed, for example, in a patient group having a good outcome (group A to be described below). The optimal range R may be set so that the average value and the variance value are obtained for each of the patient groups having the average outcome (group B to be described below), the patient group having the poor outcome (group C to be described below), and the patient group having the good outcome (group A to be described below), and the distribution is within a range of ±2σ or 80% CV. The value may be held and used as a database or may be calculated every time. Also, guidelines or evidence values may be used.

Furthermore, the output control function 124A may be performed to display a plurality of treatment methods applicable to the target patient P1 while comparing the treatment methods on the basis of the guidelines, evidence, a disease group, and a predetermined number of high-level related attribute factors.

### DISPLAY EXAMPLE 3

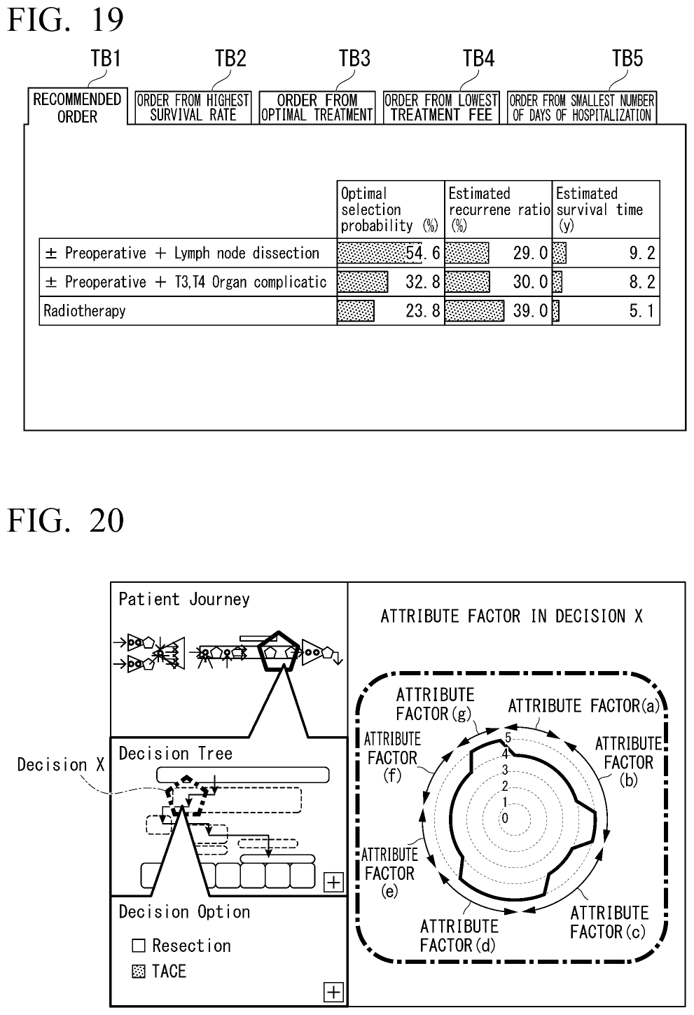

FIG. 19 shows an example in which a treatment method is displayed. As shown, the output control function 124A may be performed to provide a tab TB selectable by a user and display a treatment method for each tab TB. For example, the treatment methods may be displayed on a tab TB1 in the “recommended order,” the treatment methods may be displayed on a tab TB2 in the “order from a highest survival rate,” the treatment methods may be displayed on a tab TB3 in the “order from optimal treatment,” the treatment methods may be displayed on a tab TB4 in the “order from lowest treatment fee,” and the treatment methods may be displayed on a tab TB5 in the “order from the smallest number of days of hospitalization.” The recommended order is the order in which the order determined by other tabs is comprehensively balanced.

Furthermore, the output control function 124A may display what type of decision is being made in what type of scene in the patient journey in an entire map and may visualize and display a current position in the entire map.

The patient journey is information representing full particulars of the past, present, and/or future medical examination and treatment practices of each patient. More specifically, the patient journey is information in which medical examination and treatment practices applied to each patient in the past, medical examination and treatment practices currently being applied to the patient, or medical examination and treatment practices to be applied to the patient in the future are structured in association with the patient information of each patient. The patient information is information relating to a patient generated by the medical examination and treatment practices and includes, for example, the attribute factors described above, examination results, nursing records, and medical images (such as X-ray images and CT images).

### DISPLAY EXAMPLE 4

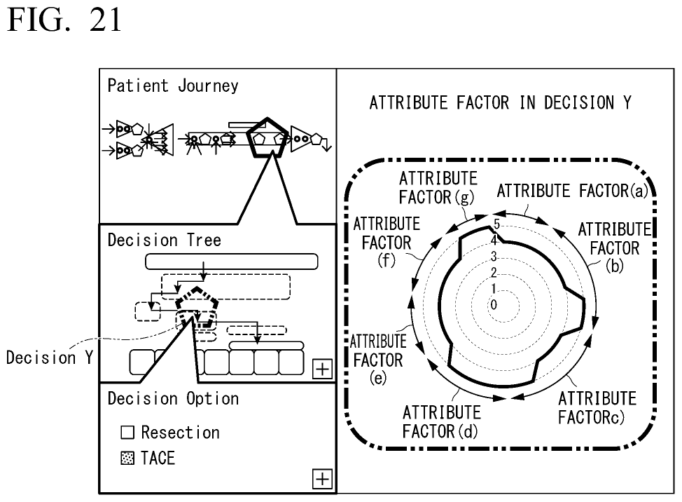

FIGS. 20 and 21 are diagrams showing examples in which the entire map is displayed. As shown, the output control function 124A may be performed to display a decision tree in the patient journey of the target patient P1 and change a related attribute factor to be displayed for each decision of the decision tree. For example, the output control function 124A may be performed to display only the related attribute factors contributing to “Decision X” in the case of “Decision X” as shown in FIG. 20 and display only the related attribute factors contributing to “Decision Y” in the case of “Decision Y” as shown in FIG. 21. In this case, the related attribute factors may be displayed as a radar chart in which the degrees of the attribute factors are graded in five steps from 0 to 5.

According to the above-described second embodiment, the medical information processing device 100 acquires a group of attribute factors relating to the disease of the target patient P1. The medical information processing device 100 identifies related attribute factors which are attribute factors highly relevant to diseases in a group of attribute factors. Also, the medical information processing device 100 outputs information based on the related attribute factor via the output interface 113.

For example, it is said that there are 60 to 80 attribute factors relating to the medical examination and treatment of cancer. These many attribute factors are present in information systems of different medical departments, for example, such as radiology and oncology, and cannot be covered by electronic medical records alone. Therefore, there is a situation where it is not possible to manage all attribute factors. Furthermore, there are a large number of attribute factors and there are various systems capable of retrieving the attribute factors. It takes a long time to examine all the attribute factors and it is easy to overlook them. Also, focusing on selecting the appropriate treatment method may overlook the relationship with patient outcomes.

In the second embodiment, to solve this problem, because related attribute factors highly relevant to diseases can be identified in a group of attribute factors relating to the disease of a target patient P1 and information based on the related attribute factors can be output, it is possible to select a treatment method of improving the outcome of a patient while increasing the reliability and efficiency of collecting attribute factors relating to the disease.

### THIRD EMBODIMENT

A third embodiment will be described below. The third embodiment is different from the second embodiment in that when a certain treatment method is applied to a target patient P1, the treatment fee of the treatment method is estimated and the treatment fee is output. The differences from the second embodiment are described mainly and the description of content identical to that of the second embodiment is omitted. In the description of the third embodiment, parts identical to those of the second embodiment are denoted by the same reference signs.

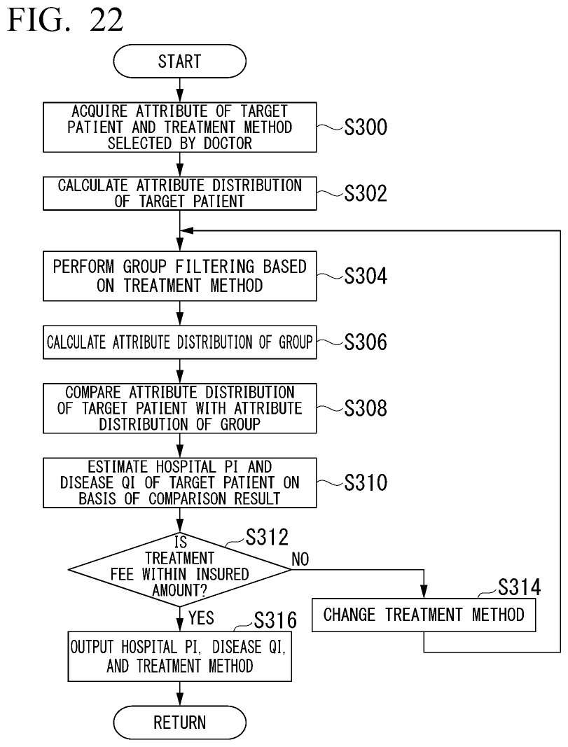

FIG. 22 is a flowchart showing a flow of a series of processing steps of processing circuitry 120A according to the third embodiment. First, an acquisition function 121A is performed to acquire attribute factors of the target patient P1 and information indicating a treatment method selected by a medical worker P2 from a terminal device 10 via a communication interface 111 (step S300).

For example, an output control function 22 of the terminal device 10 is performed to cause a display 13a to display a plurality of treatment methods applicable to the target patient P1. A medical worker P2 (particularly, a doctor) selects one or more treatment methods from among a plurality of treatment methods displayed on the display 13a and inputs a selection result to an input interface 12. The communication control function 23 is performed to transmit a result of selecting the treatment method input to the input interface 12 by the medical worker P2 to a medical information processing device 100 via a communication interface 11. Accordingly, the acquisition function 121A of the medical information processing device 100 is performed to acquire the result of selecting the treatment method selected by the medical worker P2 from the terminal device 10 via the communication interface 111.

Subsequently, an estimation function 122A is performed to calculate an attribute distribution quantitatively expressing the attribute factor of the target patient P1 acquired in the acquisition function 121A (step S302). The attribute distribution of the target patient P1 is one example of a “first distribution.”

For example, the estimation function 122A is performed to convert each of a plurality of attribute factors of the target patient P1 into a quantitative value in accordance with guidelines determined by an administrative organ or the like, medical examination and treatment standards determined by each medical institution, or the like and calculate the quantified attribute factors as a distribution. The estimation function 122A may be performed to quantify attributes of the target patient P1 using a predetermined database or quantify attributes of the target patient P1 using machine learning (deep learning or the like).

For example, the attribute distribution may be represented as a radar chart in which the degree of each attribute factor is graded in five steps from 0 to 5. That is, the values of the attribute factors may be normalized so that the minimum value becomes 0 and the maximum value becomes 5 and then may be expressed as a distribution like a radar chart.

Also, the attribute distribution is not limited to the radar chart. For example, the attribute distribution may be represented by other statistical diagrams such as histograms, stacked graphs, and heat maps. The number of steps of the attribute is not limited to 5 and may be 4 or less or 6 or more.

Subsequently, the estimation function 122A is performed to filter a population including various patients on the basis of a result of selecting the treatment method acquired in the acquisition function 121 (step S304).

The population may include, for example, a plurality of patients who were treated in the past under the medical worker P2 who is trying to treat the target patient P1 or under a medical institution where the medical worker P2 works. Medical institutions include hospitals, clinics, and other facilities where medical care is provided. The population may be a patient population in medical statistics. The population can be stratified into a plurality of groups (also called classification, grouping, or clustering) on the basis of a hospital performance index (PI) and/or a disease quality index (QI), which is a type of outcome.

The hospital PI is an index value relating to the time or economic cost spent by each patient in the population and is, for example, the number of days of hospitalization, a treatment fee, or the like. From another aspect, the hospital PI is an index value of the time or economic cost spent by the medical institution, and, for example, is the number of days of hospitalization, a medical examination and treatment fee, or the like.

Disease QI is an index value for measuring a treatment effect indicating how well a patient's disease has been treated when the patient in the population was examined and treated in accordance with a treatment method. For example, when the patient has cancer, the disease QI may be 5-year survival, the number of days of postoperative hospital stay, a recurrence rate, a cancer survival rate, or a percentage of breast-conserving surgery. Also, when the disease of the patient is acute myocardial infarction, the disease QI may be an average number of days of hospital stay or the like. Also, in the case where the patient has diabetes, the disease QI may be the improvement rate of hemoglobin A1c (HbA1c), the number of referrals of the patient, the number of reverse referrals of the patient, or the like. When the disease of the patient is pneumonia, the disease QI may be an average number of days of hospital stay or an initial treatment success rate.

For example, if the treatment method selected by the medical worker P2 is a method of “AAA,” the estimation function 122A is performed to extract a plurality of patients to which the treatment method “AAA” was previously applied from the population.

Subsequently, the estimation function 122A is performed to stratify the plurality of patients (hereinafter referred to as samples) extracted from the population on the basis of the treatment method into a plurality of groups and calculate the attribute distribution of each stratified group (step S306). The attribute distribution of each group is an example of a “second distribution.”

First, in order to calculate the attribute distribution of each group, the estimation function 122A is performed to select, as a hospital PI and/or a disease QI, one or more index values relating to the treatment method selected by the medical worker P2 from among a plurality of index values for measuring the treatment effect.

Also, the estimation function 122A is performed to stratify samples into a plurality of groups on the basis of the selected hospital PI and/or disease QI and calculate the attribute distribution of each group. For example, if a treatment method called “AAA” is unique to cancer, the estimation function 122A is performed to select an index value such as cancer-related “recurrence rate” or “cancer survival rate” as the disease QI, and stratify the samples into a plurality of groups on the basis of the disease QI relating to the cancer.

Also, the estimation function 122A may be performed to execute a stratification process at another timing different from that of the present flowchart. The other timing may be, for example, a day off from a medical institution or at night when there are relatively few patients to be examined and treated. That is, when the process of the present flowchart is started, the samples may be in a state in which they are already stratified into a plurality of groups. In this case, the estimation function 122A only needs to select a group suffering from the same disease as the target patient P1 and to which a treatment method identical to the treatment method scheduled to be applied to the target patient P1 was applied in the past from among the plurality of already stratified groups.

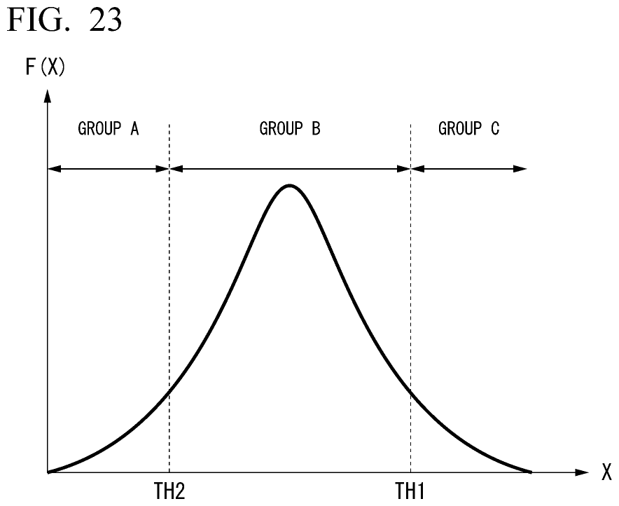

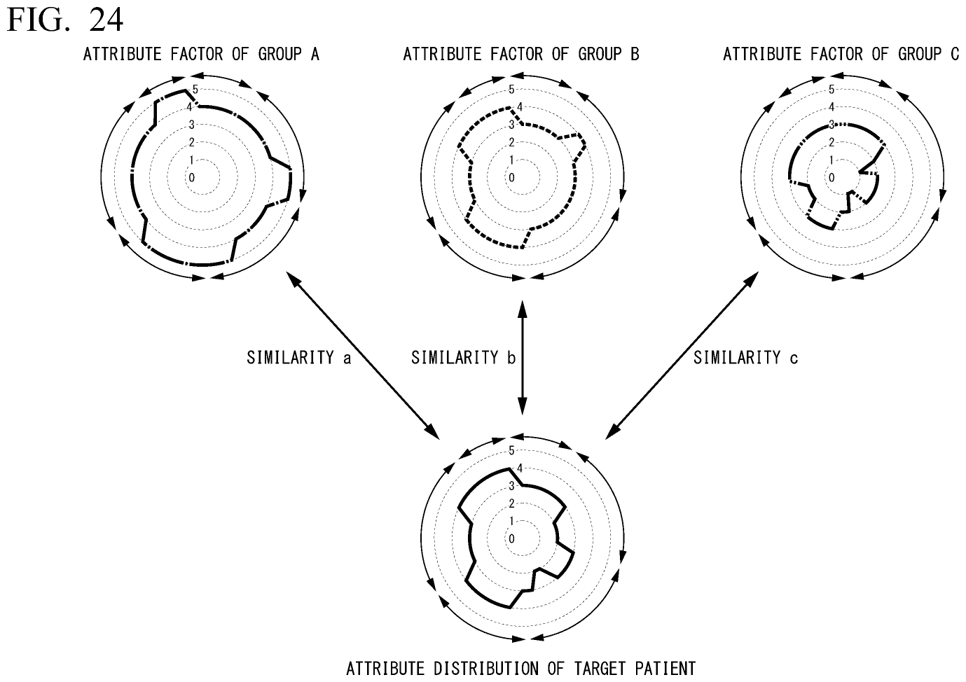

FIG. 23 is a diagram for describing the stratification process. As shown, for example, the estimation function 122A is performed to calculate a probability density distribution F(X) of a population when a hospital PI or a disease QI is defined as a probability variable X. Also, the estimation function 122A is performed to stratify the population into a plurality of groups according to a certain criterion on the probability density distribution F(X).

For example, the estimation function 122A may be performed to classify a group in which the probability variable X is less than a second threshold value TH2 as group A, classify a group in which the probability variable X is greater than or equal to the second threshold value TH2 and less than a first threshold value TH1 as group B, and classify a group in which the probability variable X is greater than or equal to the first threshold value TH1 as group C. Each of the first threshold value TH1 and the second threshold value TH2 may be, for example, a fixed value decided on the basis of a medical statistical result or a guideline, or may be a reference value from or to which a certain margin is added or subtracted with respect to a national average, an average in each medical institution, or the like. Each of the first threshold value TH1 and the second threshold value TH2 may be a standard deviation such as ±1σ, ±2σ, or ±3σ. The number of threshold values may be not limited to two and may be one or three or more. That is, the number of groups may be two or four or more.

For example, when the cancer recurrence rate as the disease QI has been selected, the estimation function 122A is performed to extract cancer patients or patients to which cancer treatment methods are applied from the population in a filtering process and calculate the probability density distribution F(X) with the cancer recurrence rate of a set of extracted patients (i.e., a sample group) as a probability variable X. Also, the estimation function 122A is performed to stratify the population into, for example, three groups A, B, and C on the probability density distribution F(X) for the cancer recurrence rate. In this case, group A is a group with a lower cancer recurrence rate, group B is a group with a higher cancer recurrence rate than group A, and group C is a group with a higher cancer recurrence rate than group B. That is, group A is a group having the most improved outcome after treatment is applied, group B is a group having the second-most improved outcome after group A, and group C is a group having the least improved outcome.

The estimation function 122A is performed to calculate an attribute distribution of each group when the population is stratified into a plurality of groups. For example, the estimation function 122A is performed to average the attribute distributions of a plurality of patients included in each group and uses an average of the attribute distributions as the attribute distribution of each group. Specifically, when group A includes 100 patients, the estimation function 122A is performed to average the attribute distributions of the 100 patients and set an average of the attribute distributions of the 100 patients as an attribute distribution of group A. The estimation function 122A may be performed to similarly calculate the attribute distribution of each group by averaging the attribute distributions of a plurality of patients with respect to other groups such as groups B and C.

Returning to the flowchart description, the estimation function 122A is subsequently performed to compare the attribute distribution of the target patient P1 with the attribute distribution of each group (step S308).

FIG. 24 is a diagram for describing an attribute distribution comparison process. For example, it is assumed that there are attribute distributions of three groups, i.e., group A, group B, and group C. In this case, the estimation function 122A is performed to compare the attribute distribution of the target patient P1 with the attribute distribution of each of groups A, B, and C and calculate similarity between the attribute distributions.

For example, when the attribute distribution is a chart showing a shape feature like a radar chart, the estimation function 122A is performed to calculate, as similarity a, similarity between a graphic shape of the attribute distribution of the target patient P1 and a graphic shape of the attribute distribution of group A. Likewise, the estimation function 122A is performed to calculate similarity between a graphic shape of the attribute distribution of the target patient P1 and a graphic shape of the attribute distribution of group B as similarity b and calculate similarity between a graphic shape of the attribute distribution of the target patient P1 and a graphic shape of the attribute distribution of group C as similarity c. The similarity becomes larger when the two attribute distributions to be compared are closer to similar shapes. Also, when the attribute distribution is a diagram in which features of colors and shades appear such as a heat map, the estimation function 122A may be performed to calculate a distance between the colors and shades (what is called a color difference) of the two attribute distributions to be compared as similarity. Specifically, the estimation function 122A may be performed to calculate a color histogram of each attribute distribution and calculate the similarity between the two attribute distributions from the Euclidean distance and the cosine similarity of the color histogram.

Returning to the flowchart description, the estimation function 122A is subsequently performed to estimate an index value of at least one of the hospital PI and the disease QI of the target patient P1 or index values of both on the basis of a comparison result between the attribute distribution of the target patient P1 and the attribute distribution of each group (step S310).

For example, the estimation function 122A is performed to estimate a hospital PI used when a group having highest similarity in the attribute distribution among a plurality of groups compared with the attribute distribution of a target patient P1 is stratified as a hospital PI relating to the target patient P1 and estimate a disease QI used when the group having the highest similarity in the attribute distribution is stratified as a disease QI relating to the target patient P1.

For example, in FIG. 24, it is assumed that the similarity b is highest. In this case, the estimation function 122A is performed to estimate the recurrence rate of cancer used when group B is stratified from the population as the disease QI of the target patient P1. Group B is a group having a cancer recurrence rate greater than or equal to a second threshold value TH2 and less than a first threshold value TH1. Thus, a value estimated as the cancer recurrence rate of the target patient P1 ranges from the second threshold value TH2 to the first threshold value TH1.

Likewise, when a hospital PI called “treatment fee” is used when group B is stratified from a population, the estimation function 122A estimates the “treatment fee” of group B used as the hospital PI of the target patient P1. Group B is also a group in which the “treatment fee” is greater than or equal to the second threshold value TH2 and less than the first threshold value TH1. Therefore, the “treatment fee” capable of being charged to the target patient P1 when the treatment method selected in the processing of S200 is applied to the target patient P1 is estimated as a range from the second threshold value TH2 to the first threshold value TH1.

Subsequently, the estimation function 122A determines whether or not the estimated treatment fee of the target patient P1 is within a range of the insured amount (step S312).

When the estimated treatment fee of the target patient P1 is outside of the range of the insured amount, the estimation function 122A reselects a treatment method that has not been selected as a new treatment method (step S314) and returns the process to 5204. Thus, it is possible to estimate the “treatment fee” which can be charged to the target patient P1 again when the reselected treatment method has been applied to the target patient P1.

On the other hand, when the estimated treatment fee of the target patient P1 is within the range of the insured amount, the output control function 124A outputs the hospital PI (for example, a treatment fee) and the disease QI (for example, a survival rate) of the target patient P1 estimated in the processing of S210 and a treatment method which is assumed to be applied to the target patient P1 when these index values are estimated via an output interface 113 (step S316). The output control function 124A may be performed to transmit the information to the terminal device 10 via the communication interface 111. Thereby, the process of the present flowchart ends.

According to the above-described third embodiment, the medical information processing device 100 filters a population according to a treatment method selected by a medical worker P2 and estimates a hospital PI and/or a disease QI of a target patient P1 on the basis of a result of comparison between the attribute distribution of a group of samples stratified in filtering and the attribute distribution of the target patient P1. Furthermore, the medical information processing device 100 determines whether or not the treatment fee of the target patient P1 estimated as the hospital PI is within the range of the insured amount, and outputs the hospital PI, the disease QI, and the treatment method when the treatment fee is within the range of the insured amount. Thereby, patients and their family members can be allowed to select a treatment method with a low financial burden.

### MODIFIED EXAMPLES OF SECOND EMBODIMENT AND THIRD EMBODIMENT

Modified examples of the second embodiment and the third embodiment (other embodiments) will be described below. Although a case where the terminal device 10 and the medical information processing device 100 are mutually different devices in the second embodiment and the third embodiment has been described, the present invention is not limited thereto. For example, the terminal device 10 and the medical information processing device 100 may be one integrated device. For example, the processing circuitry 20 of the terminal device 10 may further include an estimation function 122A and an identification function 123A provided in the processing circuitry 120A of the medical information processing device 100 in addition to the acquisition function 21, the output control function 22, and the communication control function 23. In this case, the terminal device 10 can perform the above-described processes of various types of flowcharts in a stand-alone (offline) way.

Although a case where the processes of the flowcharts shown in FIG. 15 and FIG. 22 are executed only by the medical information processing device 100 have been described, the present invention is not limited thereto. For example, the processes of these flowcharts may be partially executed by the terminal device 10.

While several embodiments of the present invention have been described above, these embodiments have been presented by way of example only, and are not intended to limit the scope of the inventions. These embodiments may be embodied in a variety of other forms. Various omissions, substitutions, and combinations may be made without departing from the spirit of the inventions. The inventions described in the accompanying claims and their equivalents are intended to cover such embodiments or modifications as would fall within the scope and spirit of the inventions.

What is claimed is:

1. A medical information processing device comprising:
acquiring, by processing circuitry, a first attribute factor group which is a plurality of attribute factors relating to a disease of a target patient and which is a plurality of attribute factors at a first timing;
estimating, by the processing circuitry, a second attribute factor group which is a plurality of attribute factors at a second timing after the first timing on the basis of the first attribute factor group; and
outputting, by the processing circuitry, information including the first attribute factor group and the second attribute factor group via an output interface.

2. The medical information processing device according to claim 1, wherein the second timing is a timing after disease treatment was performed on the target patient.

3. The medical information processing device according to claim 2, wherein the treatment on the target patient is decided on the basis of treatment results of other patient groups suffering from the same disease as the target patient.

4. The medical information processing device according to claim 3, wherein the treatment on the target patient is decided on the basis of treatment results of other patients whose previous treatment outcomes were improved among the other patient groups.

5. The medical information processing device according to claim 1,
wherein the processing circuitry stratifies the other patient groups into a plurality of groups on the basis of outcomes of the other patient groups suffering from the same disease as the target patient and calculates an attribute distribution quantitatively expressing an attribute factor group which is the plurality of attribute factors of each group, and
wherein the processing circuitry extracts a related attribute factor that is an attribute factor having a higher degree of influence on at least one of selection of a disease treatment method and improvement of the outcome than the other attribute factors from among the attribute factor groups on the basis of a result of comparing a first attribute distribution that is the attribute distribution of a first group with a second attribute distribution that is the attribute distribution of a second group having a better outcome than the first group.

6. The medical information processing device according to claim 5,
wherein the first attribute factor group includes a control factor that is the attribute factor capable of being controlled by the target patient and a non-control factor that is the attribute factor incapable of being controlled by the target patient, and
wherein the processing circuitry calculates a third attribute distribution that is the attribute distribution of the target patient and estimates the second attribute factor group on the basis of the first attribute factor group in which the control factor is adjusted so that the third attribute distribution is close to the second attribute distribution.

7. A medical information processing device comprising:
acquiring, by processing circuitry, a plurality of attribute factors relating to a disease of a target patient;
identifying, by the processing circuitry, a related attribute factor that is an attribute factor highly relevant to the disease among the plurality of attribute factors; and
outputting, by the processing circuitry, information based on the related attribute factor via an output interface.

8. The medical information processing device according to claim 7, wherein the processing circuitry identifies an attribute factor having a higher degree of influence on at least one of selection of a disease treatment method and improvement of the outcome of the target patient than the other attribute factors as the related attribute factor.

9. The medical information processing device according to claim 7, wherein the processing circuitry
compares each of the plurality of attribute factors with a reference value defined by a guideline or evidence, and
identifies the attribute factor having deviation from the reference value greater than or equal to a threshold value as the related attribute factor among the plurality of attribute factors.

10. The medical information processing device according to claim 7, wherein the processing circuitry
compares each of the plurality of attribute factors with a second attribute factor that is the attribute factor of another patient group having an improved outcome, and
identifies the attribute factor having deviation from the second attribute factor greater than or equal to a threshold value among the plurality of attribute factors as the related attribute factor.

11. The medical information processing device according to claim 7, wherein the processing circuitry estimates an outcome of the target patient on the basis of the attribute factor of the target patient.

12. The medical information processing device according to claim 11, wherein the processing circuitry
calculates a first distribution quantitatively expressing an attribute factor of the target patient,
calculates a second distribution quantitatively expressing an attribute factor of each of a plurality of groups stratified from the other patient group on the basis of outcomes of other patient groups that were previously treated, and
estimates an outcome of the target patient on the basis of a result of comparing the first distribution with the second distribution.

13. The medical information processing device according to claim 12,
wherein the outcome includes a treatment fee of a treatment method scheduled to be applied to the target patient, and
wherein the processing circuitry
determines whether or not the treatment fee estimated as the outcome of the target patient is within a range of an insured amount, and
outputs the outcome of the target patient and the treatment method scheduled to be applied to the target patient via the output interface when the treatment fee is within the range of the insured amount.

14. A medical information processing method using a computer, the medical information processing method comprising:
acquiring a first attribute factor group which is a plurality of attribute factors relating to a disease of a target patient and which is a plurality of attribute factors at a first timing;
estimating a second attribute factor group which is a plurality of attribute factors at a second timing after the first timing on the basis of the first attribute factor group; and
outputting information including the first attribute factor group and the second attribute factor group via an output interface.

15. A medical information processing method using a computer, the medical information processing method comprising:
acquiring a plurality of attribute factors relating to a disease of a target patient;
identifying a related attribute factor that is an attribute factor highly relevant to the disease among the plurality of attribute factors; and
outputting information based on the related attribute factor via an output interface.

16. A computer-readable non-transitory storage medium storing a program for causing a computer to:
acquire a first attribute factor group which is a plurality of attribute factors relating to a disease of a target patient and which is a plurality of attribute factors at a first timing;
estimate a second attribute factor group which is a plurality of attribute factors at a second timing after the first timing on the basis of the first attribute factor group; and
output information including the first attribute factor group and the second attribute factor group via an output interface.

17. A computer-readable non-transitory storage medium storing a program for causing a computer to:
acquire a plurality of attribute factors relating to a disease of a target patient;
identify a related attribute factor that is an attribute factor highly relevant to the disease among the plurality of attribute factors; and
output information based on the related attribute factor via an output interface.
